(19) United States
(12) Patent Application Publication
(10) Pub. No.: US 2024/0148918 A1
(43) Pub. Date: May 9, 2024

(54) Loading of Alginate Microspheres

(71) Applicant: Board of Regents, The University of Texas System, Austin, TX (US)

(72) Inventors: WILLIAM PHILLIPS, San Antonio, TX (US); RYAN BITAR, New Haven, CT (US)

(21) Appl. No.: 18/280,427

(22) PCT Filed: Mar. 4, 2022
(86) PCT No.: PCT/US2022/018992

§ 371 Date: Sep. 5, 2023

Related U.S. Application Data
(60) Provisional application No. 63/157,546, filed on Mar. 5, 2021.

(51) Int. Cl. A61K 51/12; A61K 9/127; A61K 9/50; A61K 45/06

(52) U.S. Cl. CPC A61K 51/1251; A61K 9/1278; A61K 9/5036; A61K 45/06; A61K 51/1234; A61K 2121/00; A61K 2123/00

(57) ABSTRACT

Certain embodiments are directed to methods for loading a liposome-containing alginate microsphere with an agent when the liposome-containing microsphere has been formed prior to loading of the liposome.

### PRIORITY PARAGRAPH

This Application is an International Application claiming priority to U.S. Provisional Patent Application Ser. No. 63/157,546 filed Mar. 5, 2021, which is incorporated herein by reference in its entirety.

### STATEMENT REGARDING FEDERALLY FUNDED RESEARCH

None.

### BACKGROUND

Hepatocellular Carcinoma (HCC) is the most common type of liver cancer. It is the sixth most common type of cancer and third most common cause of cancer mortality. HCC is particularly aggressive and has a poor survival rate (five-year survival of <5%) and therefore remains an important public health issue worldwide (GlobalData Intelligence Center-Pharma, URL pharma.globaldata.com/HomePage, 2019). HCC is most commonly found in liver exhibiting cirrhosis, or scarring of the liver, which can be caused by many factors including Hepatitis B infections, Hepatitis C infections, chronic alcohol abuse, and aflatoxins commonly found fungi that can grow on certain crops such as corn. HCC is also found to be more common in males by a 2.4:1 ratio compared to females (Balogh et al., J Hepatocell Carcinoma 3:41-53, 2016).

The primary means of treating HCC without cirrhosis is removing the tumor by surgery (resection). However a tumor may not be deemed resectable if the patient already has impaired liver function, the tumor has spread to multiple locations or is too large, or if too little of the patient's liver would remain after resection to allow for liver function post-surgery. For patients with cirrhosis, the best treatment is a liver transplant, however due to the shortage of donor organs; the wait time for patients who meet the criteria for transplant is over 2 years.

For unresectable HCC several other nonsurgical options are available that attempt to reduce the size or number of tumors to delay disease progression and to improve patient indicators to allow for resection. The most common procedure is transarterial chemoembolization, in which the one of the two main blood vessels, the hepatic artery, is blocked (embolized) to cut off the blood supply of the tumor. Prior to embolization, a chemotherapeutic agent is injected into the artery to deliver it preferentially to the tumor cells. This approach leaves the hepatic portal vein intact and is therefore thought to preserve the health of non-tumor liver cells that mainly depend on it for blood supply. Recently, the use of beads that release chemotherapeutic agents over time have been suggested to increase the effectiveness of these treatments.

Similarly, transarterial radioembolization uses the same types of particles to block the blood supply of the tumor; however, instead of chemotherapeutic agents, the particles rely on radiation given off by isotopes such as Yttrium-90 (Y-90) embedded in the particles (microspheres) that are delivered to the tumor. A variant on this procedure, known as percutaneous local ablation, follows the radioembolization with multiple days of direct injections of ethanol to the tumor.

Lastly, there is microwave ablation that uses electromagnetic waves with frequencies greater than 900 kHz to heat the tumor to a temperature higher than 100° C. This allows for a faster and more uniform ablation of the tumor, but studies have yet to show any statistical difference in efficiency compared to radioembolization.

The standard of care for patients with HCC considered too advanced for resection or localized ablation is systemic chemotherapy. The only treatment that has shown an improvement in mean survival of treatment groups is Bayer's Nexavar (sorafenib), which only prolongs survival by three months. Thus, there is a need for additional treatment options for HCC and other cancers.

### SUMMARY OF THE INVENTION

A current limitation associated with methods of producing Liposomes in Alginate Microsphere (LAMs) is that the LAMs are radiolabeled prior to incorporation into alginate microspheres resulting in inefficient loading and additional processing (e.g., filtration, etc.) of the loaded LAMs. Certain embodiments described herein provide a solution to the current problems associated with loading liposomes prior to forming LAMs. These embodiments are directed to methods of loading the liposomes after formation of LAMs, i.e., post-manufacture loading or post-loading. The post-manufacture radiolabeled LAMs can be used in delivery of chemotherapeutic and radionuclide microspheres.

Certain embodiments are directed to methods of post-loading the LAMs in which pH-gradient liposomes are encapsulated in microspheres. The LAMs can be optimized to desired size, packaged, and stored. When needed the LAMs can be loaded, for example loaded with a radiolabel, radiotherapeutic, and/or diagnostic agents. The after-production labeling or loading can be performed on-site just prior to their clinical use.

Certain embodiments are directed to methods for post-manufacture loading of liposome-containing polysaccharide microspheres comprising contacting a microsphere containing a plurality of liposomes with a loading complex comprising a therapeutic/diagnostic agent or a therapeutic/diagnostic agent couple to a loading agent, wherein the therapeutic/diagnostic agent or the therapeutic/diagnostic agent/loading agent complex or conjugate is retained in liposome. In certain aspects, the liposome-containing microspheres are suspended in an appropriate buffer. The buffer can be a saline buffer at a pH of between 6.5 and 7.5. In certain aspects, the microsphere is a hydrogel microsphere, such as an alginate microsphere. The therapeutic agent can be a chemotherapeutic agent or a radiotherapeutic agent. In certain aspects the chemotherapeutic agent is a taxane, epothilones, anthracycline (e.g., doxorubicin) or vinca alkaloid. In certain aspects the radiotherapeutic agent is ¹³¹I, ⁹⁰Y, ^{99m}Tc, ¹⁷⁷Lu, ¹⁸⁶Re, ¹⁸⁸Re, ¹²⁵I, ¹²³I, or any combination thereof. In other aspects the radiotherapeutic agent can be one or more of Bismuth-213, Cesium-131, Chromium-51, Cobalt-60, Dysprosium-165, Erbium-169, Holmium-166, Iodine-125, Iodine-131, Iridium-192, Iron-59, Lead-212, Lutetium-177, Molybdenum-99, Palladium-103, Phosphorus-32, Potassium-42, Radium-223, Rhenium-186, Rhenium-188, Samarium-153, Scandium-47, Selenium-75, Sodium-24, Strontium-89, Technetium-99m, Thorium-227, Xenon-133, Ytterbium-169, Ytterbium-177, Yttrium-90, Actinium-225, Astatine-211, Bismuth-212, Carbon-11, Fluorine-18, Nitrogen-13, Oxygen-15, Cobalt-57, Copper-64, Copper-67, Gallium-67, Gallium-68, Germanium-68, Indium-111, Iodine-123, Iodine-124, Krypton-81m, Rubidium-82, Strontium-82, and/or Thallium-201. In certain aspects, the loading agent or therapeutic agent is an amphipathic base or acid. In particular aspects, the loading agent is BMEDA.

Certain embodiments are directed to a kit for post-loading a hydrogel microsphere comprising (i) a container of hydrogel microspheres or liposome loaded microspheres and (ii) a loading agent. The kit can include other buffers or reagents need for the loading process, as well as other components to isolated loaded microspheres from unload agents.

Certain embodiments are directed to a liposome-containing microsphere, wherein the loading efficiency of a therapeutic agent in the liposome is 10 to 90%. In certain aspects the loading efficiency is at least 10%, at least 20%, at least 30%, at least 40%, at least 50%, at least 60%, at least 70%, at least 80%, or at least 90%. The loading efficiency can be between 10% and 100%, 20% and 100%, 30% and 100%, 40% and 100%, 50% and 100%, 60% and 100%, 70% and 100%, 80% and 100%, 90% and 100%, 10% and 90%, 20% and 90%, 30% and 90%, 40% and 90%, 50% and 90%, 60% and 90%, 70% and 90%, 80% and 90%, 10% and 80%, 20% and 80%, 30% and 80%, 40% and 80%, 50% and 80%, 60% and 80%, or 70% and 80%.

In certain aspects the hydrogel microsphere is a polysaccharide microsphere. The polysaccharide microsphere can be an alginate microsphere. In certain aspects the liposome includes sphingolipids, ether lipids, sterols, phospholipids, phosphoglycerides, or glycolipids.

In certain aspects an imaging agent is 99m Tc. The therapeutic agent can be a chemotherapeutic agent or a radiotherapeutic agent. The chemotherapeutic agent can be a taxane, epothilones, anthracycline (e.g., doxorubicin), or vinca alkaloid. The radiotherapeutic agent can be ¹³¹I, ⁹⁰Y, ¹⁷⁷Lu, ¹⁸⁶Re, ¹⁸⁸Re, ¹²⁵I, or ¹²³I or any combination thereof.

In certain aspects the loading agent is BMEDA.

Certain embodiments are directed to a liposome-containing microsphere having a specific activity of 40, 50, 60, 70, 80, 90, 100, 200, 300, 400, 500, 600, 700, 800, 900, 1000, to 2000, 3000, 4000, 5000, 6000, 7000, 8000, 9000, 10000, 15000, 20000 Bq/microsphere or more, including all values and ranges there between. In certain aspects a liposome-containing microsphere has a specific activity of at least 200, at least 500, at least 1000, at least 5000, at least 10000, at least 15000, or at least 20000 Bq/microsphere.

Other embodiments are directed to methods for performing embolization therapy on a subject having a tumor, or a diagnostic or imaging procedure on a subject comprising injecting a liposome-containing microsphere described herein into the subject's vasculature, preferably tumor vasculature.

Other embodiments are directed to a liposome-containing microsphere composition for use in treating or diagnosing a condition in a subject, the liposome-containing microsphere comprising a microsphere encapsulating a plurality of pH gradient liposomes encapsulating a therapeutic agent complexed with a loading agent, diagnostic agent complexed with a loading agent, or a combination thereof, wherein the loading efficiency of a therapeutic agent is 10, 20, 30, 40, 50, 60, 70, 80, 90, to 100%, including all ranges and values there between. In certain aspects the therapeutic agent or diagnostic agent is one or more of Bismuth-213, Cesium-131, Chromium-51, Cobalt-60, Dysprosium-165, Erbium-169, Holmium-166, Iodine-125, Iodine-131, Iridium-192, Iron-59, Lead-212, Lutetium-177, Molybdenum-99, Palladium-103, Phosphorus-32, Potassium-42, Radium-223, Rhenium-186, Rhenium-188, Samarium-153, Scandium-47, Selenium-75, Sodium-24, Strontium-89, Technetium-99m, Thorium-227, Xenon-133, Ytterbium-169, Ytterbium-177, Yttrium-90, Actinium-225, Astatine-211, Bismuth-212, Carbon-11, Fluorine-18, Nitrogen-13, Oxygen-15, Cobalt-57, Copper-64, Copper-67, Gallium-67, Gallium-68, Germanium-68, Indium-111, Iodine-123, Iodine-124, Krypton-81m, Rubidium-82, Strontium-82, and/or Thallium-201.

Other embodiments are directed to a liposome-containing microsphere produced by the methods described herein.

Some advantages of post-loading LAMs include: (1) Ability to image with high quality. 99mTc or rhenium-188 can be imaged with ideal photon energies. This is a big advantage compared to Y-90 therapeutic agents which do not have a gamma photon and only their beta particle produced photons can be imaged. (2) Great improvement over rhenium-188 lipiodol which is not stable in vivo and which results in significant lung and kidney activity. (3) Post-loaded LAMs can be produced within 2 hours of ordering. Typical Y-90 microspheres can require ordering two weeks ahead of time. (4) LAMs are biodegradable and allow the natural clearance of rhenium through the kidneys with no bone avidity. Y-90 resin microspheres are not biodegradable and can release Y-90 which is taken up by the bones. Certain Y-90 microspheres are made of glass and are not biodegradable. (5) Biodegradability enables retreatment because of the subject clearing of some agents. (6) Another advantage is that the microsphere for pre-dosimetry imaging with 99mTc is exactly the same size as the therapeutic microsphere, allowing for more accurate pre-dosimetry assessment. This is not true with Y-90 pre-dosimetry which is done with 99mTc-macroaggregated albumin of a very different size distribution.

The liposome component of the LAM can encapsulate a variety of useful substances. Substances of note that can be encapsulated in liposomes incorporated into alginate microspheres include radiotherapeutics (e.g., rhenium-188), radiolabels (e.g., technetium-99m), chemotherapeutics (doxorubicin), magnetic particles (e.g., 10 μm iron nanoparticles), and radio-opaque material (e.g., iodine contrast). In certain aspects, rhenium-188 liposomes in alginate microspheres (Rhe-LAMs) can be used for treatment of liver tumors, specifically hepatocellular carcinoma (HCC). In a more particular aspect HCC treatment can be through radioembolization, where the microspheres block the blood supply to the tumor from the artery, while the rhenium-188 also delivers a high dose of radiation that is primarily targeted to the cancer cells.

As used herein, a “liposome” refers to a vesicle consisting of an aqueous core enclosed by one or more phospholipid layers. Liposomes may be unilamellar, composed of a single bilayer, or they may be multilamellar, composed of two or more concentric bilayers. Liposomes range from small unilamellar vesicles (SUVs) to larger multilamellar vesicles (LMVs). LMVs form spontaneously upon hydration with agitation of dry lipid films/cakes which are generally formed by dissolving a lipid in an organic solvent, coating a vessel wall with the solution and evaporating the solvent. Energy is then applied to convert the LMVs to SUVs, LUVs, etc. The energy can be in the form of, without limitation, sonication, high pressure, elevated temperatures and extrusion to provide smaller single and multi-lamellar vesicles. During this process some of the aqueous medium is entrapped in the vesicle. Liposomes can also be prepared using emulsion templating. Emulsion templating comprises, in brief, the preparation of a water-in-oil emulsion stabilized by a lipid, layering of the emulsion onto an aqueous phase, centrifugation of the water/oil droplets into the water phase and removal of the oil phase to give a dispersion of unilamellar liposomes. Liposomes prepared by any method, not merely those described above, may be used in the compositions and methods of this invention. Any of the preceding techniques as well as any others known in the art or as may become known in the future may be used as compositions of therapeutic agents in or on a delivery interface of this invention. Liposomes comprising phospholipids and/or sphingolipids may be used to deliver hydrophilic (water-soluble) or precipitated therapeutic compounds encapsulated within the inner liposomal volume and/or to deliver hydrophobic therapeutic agents dispersed within the hydrophobic bilayer membrane. In certain aspects the liposome comprises lipids selected from sphingolipids, ether lipids, sterols, phospholipids, phosphoglycerides, and glycolipids. In certain aspects, the lipid includes, for example, DSPC (1,2-di stearoyl-sn-glycero-3-phosphocholine).

The terms “loading”, “encapsulation”, or “entrapment” as used herein, referred to an incorporation of agents into the interior, lumen, or core of a liposome.

The terms “loading efficiency”, “entrapment efficiency” or “encapsulation efficiency” as used herein interchangeably, is referred to the fraction of incorporation of agent into the interior, lumen, or core of liposomes expressed as a percentage of the total initial amount used in the preparation.

As used herein a “loading agent” or “entrapment agent” is a moiety that is chemically altered once inside a liposome, the modification retaining the moiety within the liposome. A loading agent can be an amphipathic weak base that is non-ionized at a pH of 6 to 8 and may diffuse through the liposome membrane; however, at an acidic less than pH 6, e.g., pH of 5, loading agent is ionized and trapped in the lumen of the liposome. Loading of liposomes using gradients can be applied to agents having structural features that allow the drug to permeate and diffuse via the lipid bilayer to accumulate within the liposome yet prevent permeation and diffusion from liposomes. Amphipathic weak acids or bases fit can be used to affect this loading mechanism. Loading by pH or ion gradients requires that the loaded molecules have a logD at pH 7 in the range of −2.5 to 2.0 and pKa of ≤11 for an amphipathic weak base or pKa of >3 for an amphipathic weak acid. Some agents will have these groups as part of their structure while other agents can be coupled to a loading agent, e.g., chelators for metals etc. In particular aspects, the loading agent is BMEDA.

The term “hydrogel” refers to a water-containing three dimensional hydrophilic polymer network or gel in which the water is the continuous phase. In certain aspects the hydrogel is an alginate hydrogel.

As used herein, “alginate” refers to a linear polysaccharide that can be derived from seaweed. The most common source of alginate is the species Macrocystis pyrifera. Alginate is composed of repeating units of D-mannuronic (M) and L-guluronic acid (G), presented in both alternating blocks and alternating individual residues. Soluble alginate may be in the form of monovalent salts including, without limitation, sodium alginate, potassium alginate and ammonium alginate. In certain aspects, the alginate includes, but is not limited to one or more of sodium alginate, potassium alginate, calcium alginate, magnesium alginate, ammonium alginate, and triethanolamine alginate. Alginates are present in the formula in amounts ranging from 5 to 80% by weight, preferably in amounts ranging from 20 to 60% by weight, and most preferably about 50% by weight. In certain aspects, the alginate is ultra-pure alginate (e.g., Novamatrix ultra-pure alginate). Alginate can be cross-linked using ionic gelation provided through multivalent cations in solution, e.g., an aqueous or alcoholic solution with multivalent cations therein, reacting with alginates. Multivalent cations (e.g., divalent cations, monovalent cations are not sufficient for cross-linking alginate) for use with alginates include, but are not limited to calcium, strontium, barium, iron, silver, aluminum, magnesium, manganese, copper, and zinc, including salts thereof. In certain aspects, the cation is calcium and is provided in the form of an aqueous calcium chloride solution.

In certain aspects the therapeutic or imaging agent is a chemotherapeutic, radiotherapeutic, thermotherapeutic, or a contrast agent.

In certain aspects, a radiotherapeutic agent includes a radiolabel or radiotherapeutic such as a beta emitter (¹³¹I, ⁹⁰Y, ¹⁷⁷Lu, ¹⁸⁶Re, ¹⁸⁸Re, any one of which can be specifically excluded) or gamma emitter (¹²⁵I, ¹²³I, ^{99m}Tc,), or any combination thereof. In certain aspects, the radiotherapeutic agent is 188 Re. Furthermore, the term “radiotherapeutic” may be taken to more broadly encompass any radioactively-labeled moiety, and may include any liposome or LAM associated with or comprising a radionuclide. Nuclear reactors are the source of many radioisotopes while are sourced from cyclotrons. In general, nuclear fission [reactors] produce neutron rich isotopes while neutron depleted isotopes, for example PET radionuclides are cyclotron produced [cyclotron energy ˜10-20 MeV for usual PET positron isotopes whereas single photon products usually require higher cyclotron energy [˜30 MeV]. In certain embodiments the radiotherapeutic can be a reactor radioisotope or a cyclotron radioisotope. Reactor radioisotopes can include (1) a therapeutic [Rx], both beta and alpha and low energy x-rays [for brachytherapy] and/or (2) a diagnostic [Dx], both positron and single photon. The Rx or Dx listed here are exemplary embodiments of how the radioisotopes can be used. The scope of the invention includes utilizing the radioisotopes listed here in other Rx or Dx. Reactor radioisotopes include, but are not limited to: Bismuth-213 (alpha), Cesium-131 (x-rays brachyRx), Chromium-51 (Dx), Cobalt-60 (historically EBRT now universally used for sterilizing; historically HSACo-60 for brain cancer Rx), Dysprosium-165 (beta Rx), Erbium-169 (beta Rx), Holmium-166 (beta Rx), Iodine-125 (low energy x-rays Rx brachytherapy and RIA applications), Iodine-131 (Beta Rx [fission product]; has an imaging gamma, albeit high energy), Iridium-192 (beta Rx; often in wire form for brachytherapy, e.g., prostate), Iron-59 (Dx historically iron metabolism studies), Lead-212 (alpha Rx), Lutetium-177 (Rx beta; has gamma emission for imaging), Molybdenum-99 (Dx—parent of Tc99m [fission product]), Palladium-103 (Rx low energy x-rays example of permanent implant brachytherapy), Phosphorus-32 (beta Rx; historic Rx of polycythemia vera), Potassium-42 (Dx historic measure of exchangeable K+ for coronary blood flow), Radium-223 (Rx alpha; historic brachyRx with low-energy x-rays), Rhenium-186 (beta Rx with imaging photon; historic Rx bone pain), Rhenium-188 (beta Rx; historic coronary arteries via stent), Samarium-153 (beta Rx; historic product [Quadramet] for bone pain/metastasis), Scandium-47 (beta Rx with imaging capability; —Lu-177; produced by irradiating Ca-46 to produce Ca-47 which decays to Sc-47), Selenium-75 (Dx; historic seleno-methionine for GI study), Sodium-24 (Dx historic electrolytes study), Strontium-89 (Rx bone pain and metastasis [fission product]), Technetium-99m (Dx; workhorse Dx isotope in nuclear medicine; produced in generator from Mo-99), Thorium-227 (Rx alpha; decays to Ra-223 another alpha Rx), Xenon-133 (Dx [a gas-fission product]), Ytterbium-169 (Dx; used before In-111 for CSF flow studies), Ytterbium-177 (Rx precursor of Lu-177 via Yb-176 neutron irradiation), and Yttrium-90 (Rx pure beta emitter [fission product]). Cyclotron radioisotopes include, but are not limited to: Actinium-225 (Rx alpha), Astatine-211 (Rx alpha), Bismuth-212 (Rx alpha), Carbon-11 (Dx positron/PET), Fluorine-18 (Dx positron/PET), Nitrogen-13 (Dx positron/PET), Oxygen-15 (Dx positron/PET), Cobalt-57 (Dx in-vitro Dx kits), Copper-64 (Dx positron; historic studies copper metabolism), Copper-67 (Rx beta), Gallium-67 (Dx single photon), Gallium-68 (Dx positron), Germanium-68 (Dx—parent for Ga-68 generator), Indium-111 (Dx), Iodine-123 (Dx, no beta emission), Iodine-124 (Dx positron), Krypton-81m (Dx [gas generator produced from Rb-81 at bedside T1/2=13 seconds]), Rubidium-82 (Dx positron potassium analog for perfusion imaging; generator produced at patient T1/2=75 seconds), Strontium-82 (Dx-parent for the Rb-82 generator), and Thallium-201 (Dx). The liposome or LAM may be associated with a radionuclide through a chelator, direct chemical bonding, or some other means such as a linker protein.

In certain aspects, a chemotherapeutic agent includes, but is not limited to a chemical compound that inhibits or kills growing cells and which can be used or is approved for use in the treatment of cancer. Exemplary chemotherapeutic agents include cytostatic agents which prevent, disturb, disrupt or delay cell division at the level of nuclear division or cell plasma division. Such agents may stabilize microtubules, such as taxanes, in particular docetaxel or paclitaxel, and epothilones, in particular epothilone A, B, C, D, E, and F, or may destabilize microtubules such as vinca alkaloids, in particular vinblastine, vincristine, vindesine, vinflunine, and vinorelbine. Other chemotherapies include anthracyclines such as doxorubicin, 4′-epi-doxorubicin (i.e., epirubicin), 4′-desoxy-doxorubicin (i.e., esorubicin), 4′-desoxy-4′-iodo-doxorubicin, daunorubicin and 4-demethoxydaunorubicin (i.e., idarubicin). Liposomes can be used to carry hydrophilic agents as micelles and can be used to carry lipophilic agents.

In general, the thermotherapeutic agents include a plurality of magnetic nanoparticles, or “susceptors,” of an energy susceptive material that are capable of generating heat via magnetic hysteresis losses in the presence of an energy source, such as, an alternating magnetic field (AMF). The methods described herein, generally, include the steps of administering an effective amount of a thermotherapeutic compound to a subject in need of therapy and applying energy to the subject. The application of energy may cause inductive heating of the magnetic nanoparticles which in turn heats the tissue to which the thermotherapeutic compounds were administered sufficiently to ablate tissue. In certain aspects, a thermotherapeutic agent includes, but is not limited to magnetite (Fe₃O₄), maghemite (γ-Fe₂O₃) and FeCo/SiO₂, and in some embodiments, may include aggregates of superparamagnetic grains of, for example, Co₃₆C₆₅, Bi₃Fe₅O₁₂, BaFe₁₂O₁₉, NiFe, CoNiFe, Co—Fe₃O₄, and FePt—Ag, where the state of the aggregate may induce magnetic blocking. In thermotherapy, the response of MNPs to AC magnetic field causes thermal energy to be dissipated into the surroundings, killing the tumor cells. Additionally, hyperthermia can enhance radiation and chemotherapy treatment of cancer. The term “hyperthermia”, as used herein, refers to heating of tissue to temperatures between about 40° C. and about 60° C. The term “alternating magnetic field” or “AMF”, as used herein, refers to a magnetic field that changes the direction of its field vector periodically, typically in a sinusoidal, triangular, rectangular or similar shape pattern, with a frequency of in the range of from about 80 kHz to about 800 kHz. The AMF may also be added to a static magnetic field, such that only the AMF component of the resulting magnetic field vector changes direction. It will be appreciated that an alternating magnetic field may be accompanied by an alternating electric field and may be electromagnetic in nature. In certain embodiments, the thermotherapeutic agent can be incorporated into alginate microspheres in the absence of lipids and as such form a thermotherapeutic containing alginate microsphere where the agent is not incorporated in a liposome but is incorporated in the alginate microsphere.

In certain aspects, a contrast or imaging agent includes, but is not limited to a transition metal, carbon nanomaterials such as carbon nanotubes, fullerene and graphene, near-infrared (NIR) dyes such as indocyanine green (ICG), and gold nanoparticles. Transition metal refers to a metal in Group 3 to 12 of the Periodic Table of Elements, such as titanium (Ti), vanadium (V), niobium (Nb), tantalum (Ta), chromium (Cr), molybdenum (Mo), tungsten (W), manganese (Mn), iron (Fe), ruthenium (Ru), osmium (Os), iridium (Ir), nickel (Ni), copper (Cu), technetium (Tc), rhenium (Re), cobalt (Co), rhodium (Rh), iridium (Ir), palladium (Pd), platinum (Pt), silver (Ag), gold (Au), a lanthanide such as europium (Eu), gadolinium (Gd), lanthanum (La), ytterbium (Yb), and erbium (Er), or a post-transition metal such as gallium (Ga), and indium (In). In one aspect, the imaging modality is selected from the group comprising, Positron Emission Tomography (PET), Single Photon Emission Tomography (SPECT), Computed Tomography (CT), Magnetic Resonance Imaging (MRI), Ultrasound Imaging (US), and Optical Imaging. In another aspect of the invention, the imaging modality is Positron Emission Tomography (PET). The imaging agent includes, but is not limited to a radiolabel, a fluorophore, a fluorochrome, an optical reporter, a magnetic reporter, an X-ray reporter, an ultrasound imaging reporter or a nanoparticle reporter. In another aspect of the invention, the imaging agent is a radiolabel selected from the group comprising a radioisotopic element selected from the group consisting: of astatine, bismuth, carbon, copper, fluorine, gallium, indium, iodine, lutetium, nitrogen, oxygen, phosphorous, rhenium, rubidium, samarium, technetium, thallium, yttrium, and zirconium. In another aspect, the radiolabel is selected from the group comprising zirconium-89 (⁸⁹Zr), iodine-124 (¹²⁴I) iodine-131 (¹³¹I) iodine-125 (¹²⁵I) iodine-123 (¹²³I), bismuth-212 (²¹²Bi), bismuth-213 (²¹³Bi), astatine-211 (²¹¹At), copper-67 (⁶⁷Cu), copper-64 (⁶⁴Cu), rhenium-186 (¹⁸⁶Re), rhenium-188 (¹⁸⁸Re), phosphorus-32 (³²P), samarium-153 (¹⁵³Sm), lutetium-177 (¹⁷⁷Lu), technetium-99m (^{99m}Tc), gallium-67 (⁶⁷Ga), indium-111 (¹¹¹In), thallium-201 (²⁰¹Tl) carbon-11, nitrogen-13 (¹³N), oxygen-15 (¹⁵O), fluorine-18 (¹⁸F), and rubidium-82 (⁸²Ru).

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features, and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Illustration of one example of a loaded liposome-containing microsphere, for example Re-188 loaded microsphere.

FIG. 2. Illustration of one example of the equipment and process for forming liposome-containing alginate microspheres.

FIG. 3. Illustrates one example of the mechanism for pH gradient liposome loading.

FIG. 4. Diagram generalizing post-loading of liposome-containing alginate microspheres.

FIG. 5. Illustration of one example of radiolabeling pre-made liposome-containing alginate microspheres.

FIGS. 6A, 6B, and 6C. One example of results obtained using the post-loading methods for producing a labeled liposome-containing alginate microsphere—(6A) size distribution of microspheres counted per size range by microscopic analysis, mean 49.5 microns with a standard deviation of 10.4; (6B) microsphere image; and (6C) radiolabeling efficiency by scintigraphy: Left is a scintigraph of wash and pellet (15% of the dose) of rhenium-chelate; Right is a scintigraph of wash and pellet (51% of the dose) of Rhenium-chelate in liposomes in alginate microspheres.

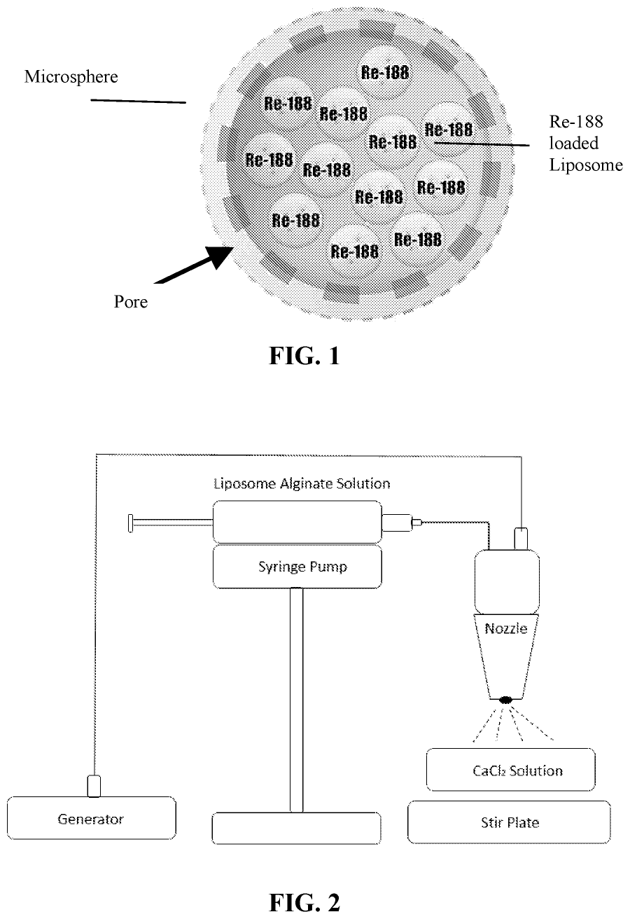

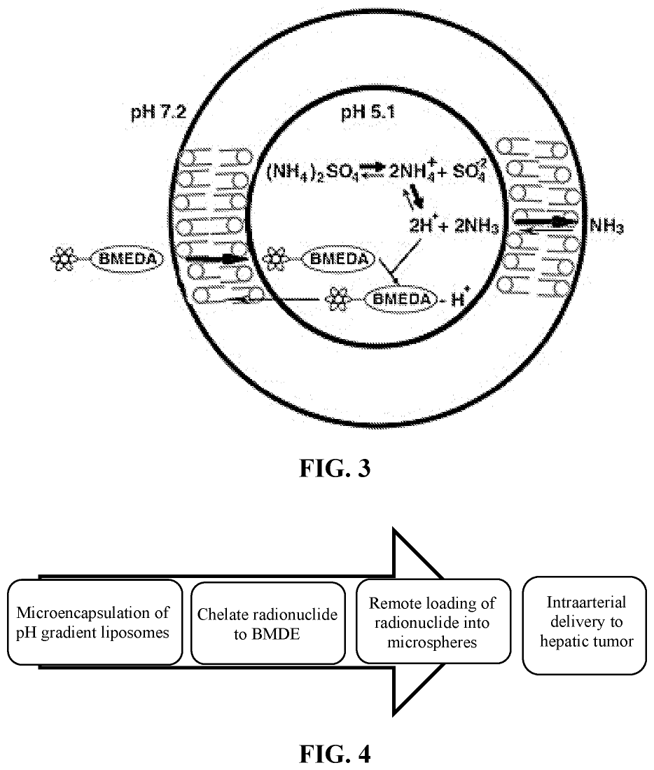

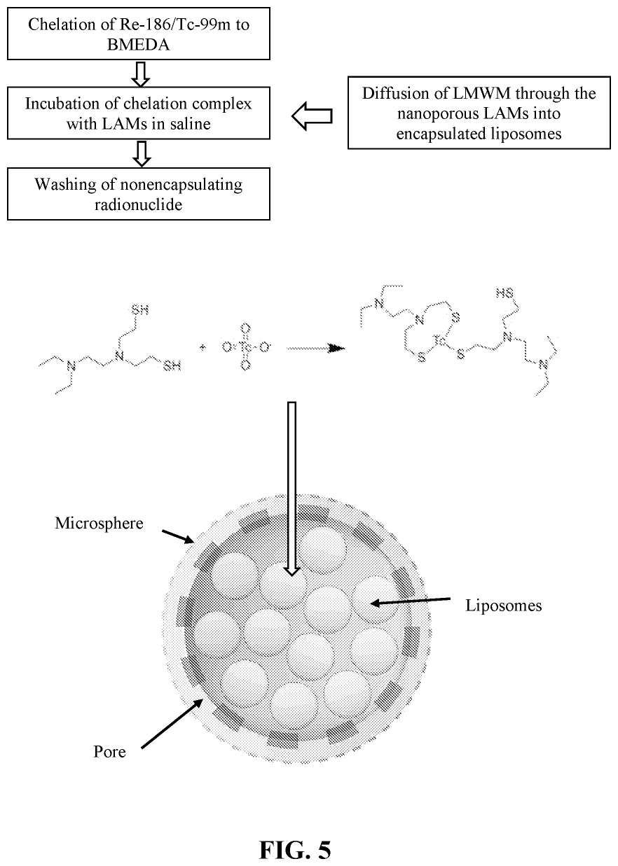

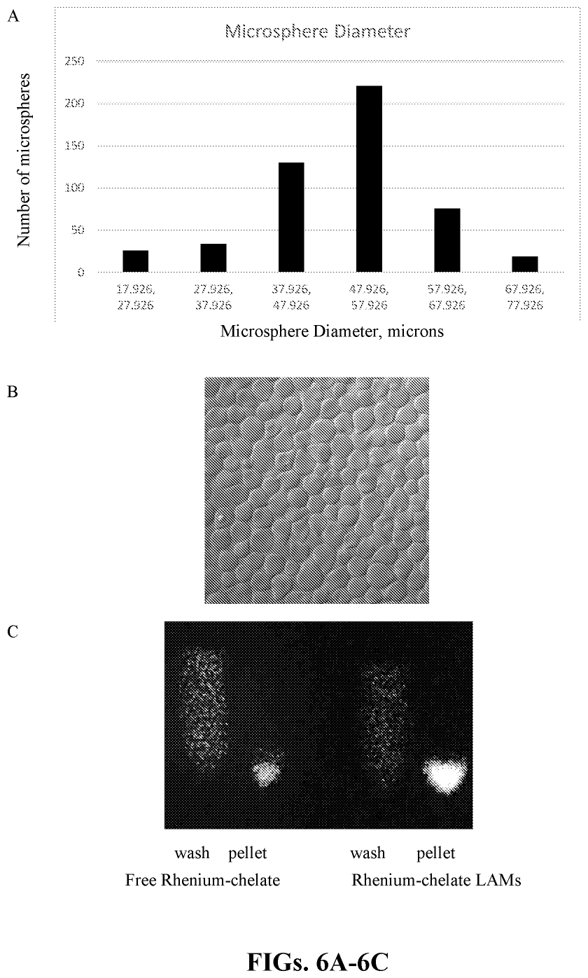

### DETAILED DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Liposome in Alginate Microspheres has potential as an agent for Transarterial Radioembolization (TARE), a common technique employed by Interventional radiologists for treating moderate stage liver tumors. The methods described herein provide a technique in which pH gradient liposomes are loaded into nanoporous microspheres forming a non-loaded LAM. The LAM can be loaded with an agent or loading complex, e.g., Tc/Re-BMEDA (a low molecular weight molecule). The loading complex has an intrinsic property which allows it to enter a liposome where it is converted and trapped in the acidic lumen of the liposome. This molecule may diffuse through the nanoporous alginate matrix and into the microencapsulated liposomes. This technique of loading a LAM post-formation greatly increases the feasibility and marketability of this agent towards the industry of radioembolization.

Described herein is an approach for post-manufacture radiolabeling of Liposomes in Alginate Microspheres (LAMs)—that is loading a liposome that is encapsulated in a microsphere, post-loading of LAMs. In certain aspects the post-manufacture labeled LAMs can be used for delivery of chemotherapeutic and radionuclide microspheres. For example, LAMS can be loaded with Tc-99m, Re-186, Re-188 or any combination thereof. Previous method have loaded these agents into liposomes prior to incorporation into alginate microspheres, pre-loading LAMs. The current application describes a method post-loading the LAMs in which pH-gradient liposomes are encapsulated in alginate microspheres prior to loading. Liposome with a pH gradient are liposomes that have a different pH on the interior to liposome as compared to the pH of the external environment. These post-loaded LAMs can be optimized to desired size. Post-loaded LAMs can also be radiolabeled in proximity to or at a location of use, e.g., just prior to clinical use.

Advantages of post-loading the microspheres include: (1) Capability to refine the alginate microspheres to the ideal size prior to post-loading with, for example, radionuclides or chemotherapeutic agents. This allows optimization of the already homogeneously sized LAM via ultrasonic nebulizer of the alginate microspheres. (2) Capability to load a higher concentration of agent, such as rhenium-188, because no filtration is required. (3) Capability to prepare a loaded LAM in a short period of time, e.g., within a few hours of notification at the local radiopharmacy and using standard radiopharmacy methodology. (4) Capability to post-load chemotherapeutic agents such as doxil into the LAMs locally, e.g., at a pharmacy or by interventional radiologists, a short time prior to use (e.g., within minutes to a few hours) which provides FDA approval advantages in that stability studies of chemotherapeutic agents, such as the most commonly used chemotherapeutic agent doxorubicin, in LAMs for months prior to their use will not be required.

Mechanisms that makes post-loading possible include the nanoporosity of alginate microspheres that allows low molecular weight molecules such as chemotherapeutic drugs and radionuclide chelation complexes to diffuse into LAMs and into the liposome component of the LAM. Once in the interior or lumen, the acidity entraps certain amphipathic bases in the liposome, loading the LAM.

Liposome formation. Construct ammonium sulfate gradient liposomes. Add phospholipids and cholesterol to a round-bottomed flask in appropriate amounts. Add chloroform or chloroform-methanol depending on lipid composition to dissolve lipids and form lipid solution. Conduct rotary evaporation on lipid solution to remove solvent and form lipid thin film. Temperature and evaporation time will vary based on lipid formulation. Desiccate lipid thin film under vacuum for at least 4 h. In certain aspects desiccation can be overnight. Rehydrate lipid thin film (e.g., 300 mM sucrose in sterile water) for injection at a predetermined total lipid concentration (e.g., 60 mM). Vortex solution and heat above lipid phase transition temperature until all lipids are in solution. Freeze lipid solution and lyophilize forming a dry powder. The dry powder is rehydrated in an appropriate buffer (e.g., ammonium sulfate in sterile water) to an appropriate total lipid concentration (e.g., 60 mM) forming a new solution. Vortex the solution vigorously and heat above lipid phase transition temperature until all lipids are in solution. Freeze the lipid solution with liquid nitrogen and then thaw in water bath set to temperature above the lipid phase transition temperature. Repeat freeze-thaw procedure for at least three cycles. Extrude liposome sample until desired particle diameter is achieved. After extrusion, final liposome product should be stored at 4° C. until needed. The liposomes can be characterized by laser light scattering particle sizing, pyrogenicity, sterility, and lipid concentration.

Microencapsulation of Liposomes in alginate microspheres. Liposomes are homogenized in an alginate solution and then fed into an ultrasonicator nozzle with microbore inserted. Briefly, a solution of ultrapure alginate can be made (concentration 3.0% w/v) at least 2 days prior to sphere production. 2 ml of the radiolabeled lipid solution is combined with 2 ml of the alginate solution and then vortexed until homogenized. The Sonotek Ultrasonicating atomizer apparatus can be set up as per FIG. 2. The generator is activated at a power of 5.0 Watts. The liposome alginate solution is fed into the nozzle at a rate of 0.5 ml/minute via syringe pump. The newly formed microdroplets descend into a stirring 20 g/L CaCl₂ dihydrate solution. The spheres are sieved to a size range of 20-70 microns. The sphere pellet is suspended in 10 ml of CaCl₂ dihydrate solution. The pH of the sphere solution was adjusted to ˜7.4.

Chelation of Tc-99m to N,N-bis(2-mercapatoethly)-N′,N′-diethylenediamine (BMEDA). The chelation of Tc-99m to BMEDA was performed as described by Goins et al. (J Liposome Res 2011, 21(1):17-27). Briefly, 3.5 μl of BMEDA and 50 mg sodium glucoheptonate are dissolved in 5.0 ml nitrogen-degassed saline in a 10 ml sterile glass serum vial. The solution is stirred for 20 min at room temperature. 65 μl of a freshly prepared 15 mg/ml stannous chloride in saline is added to the BMEDA solution. Quickly the pH of the BMEDA-GH-stannous chloride solution is adjusted to 7.0 using 50 mM sodium hydroxide. 1 ml of the adjusted solution is placed into a new vial containing 0.5 ml of 99mTc-sodium pertechnetate (dose independent). The dose is measured using a dose calibrator (Atomlab 100 Biodex Medical Systems, Shirley, New York). After gently shaking the 99mTc-BMEDA solution, it is incubated at room temperature for 20 minutes. The pH of this solution was adjusted to ˜7.4.

BMEDA and other loading moieties are an amphipathic weak base. (at pH of 7, it is non-ionized and may diffuse through the liposomes membrane; however, at pH of 5 it is ionized and is thus trapped in the lumen of the liposome due to its charge. This property is also evident in some drugs; the most well-known candidate being doxorubicin, which has already been implemented for the agent Doxil (a liposomal formulation of doxorubicin which employs the same loading mechanism as BMEDA.)

Post-Loading of Tc-99m into LAMs. The Tc-99m-BMEDA solution is mixed with the microsphere solution. The combined solution is then incubated in a water bath at 40° C. for 2 hours. Afterwards, the spheres are washed twice in calcium chloride solution to remove nonencapsulated radionuclide. Microspheres are resuspended in normal saline in preparation for intraarterial delivery.

### Hydrogel Microspheres

Methods of manufacturing hydrogel microparticles allows loading of liposomes in hydrogel microparticles. The hydrogel microparticles having liposomes encapsulated therein may be formed from a degradable hydrogel. As used herein, the term “degradable hydrogel” refers to a hydrogel having a structure which may decompose to smaller molecules under certain conditions, such as temperature, abrasion, pH, ionic strength, electrical voltage, current effects, radiation and biological means. As used in this application, the term “hydrogel” refers to a broad class of polymeric materials, that may be natural or synthetic, which have an affinity for an aqueous medium, and may absorb large amounts of the aqueous medium, but which do not normally dissolve in the aqueous medium. Generally, a hydrogel may be formed by using at least one, or one or more types of hydrogel-forming agent, and setting or solidifying the one or more types of hydrogel-forming agent in an aqueous medium to form a three-dimensional network, wherein formation of the three-dimensional network may cause the one or more types of hydrogel-forming agent to gel so as to form the hydrogel. The term “hydrogel-forming agent”, also termed herein as “hydrogel precursor”, refers to any chemical compound that may be used to make a hydrogel. The hydrogel-forming agent may comprise a physically cross-linkable polymer, a chemically cross-linkable polymer, or mixtures thereof.

Physically cross-linking may take place via, for example, complexation, hydrogen bonding, desolvation, van der Waals interactions, or ionic bonding. In various embodiments, a hydrogel may be formed by self-assembly of one or more types of hydrogel-forming agents in an aqueous medium. The term “self-assembly” refers to a process of spontaneous organization of components of a higher order structure by reliance on the attraction of the components for each other, and without chemical bond formation between the components. For example, polymer chains may interact with each other via any one of hydrophobic forces, hydrogen bonding, Van der Waals interaction, electrostatic forces, or polymer chain entanglement, induced on the polymer chains, such that the polymer chains aggregate or coagulate in an aqueous medium to form a three-dimensional network, thereby entrapping molecules of water to form a hydrogel. Examples of physically cross-linkable polymer that may be used include, but are not limited to, gelatin, alginate, pectin, furcellaran, carageenan, chitosan, derivatives thereof, copolymers thereof, and mixtures thereof.

Chemical crosslinking may take place via, for example, chain reaction (addition) polymerization, and step reaction (condensation) polymerization. The term “chemical cross-link” as used herein refers to an interconnection between polymer chains via chemical bonding, such as, but not limited to, covalent bonding, ionic bonding, or affinity interactions (e.g. ligand/receptor interactions, antibody/antigen interactions, etc.). Examples of chemically cross-linkable polymer that may be used include, but are not limited to, starch, gellan gum, dextran, hyaluronic acid, poly(ethylene oxides), polyphosphazenes, derivatives thereof, copolymers thereof, and mixtures thereof. Such polymers may be functionalized with a methacrylate group for example, and may be cross-linked in situ via polymerization of these groups during formation of the emulsion droplets in the fabrication process.

Chemical cross-linking may take place in the presence of a chemical cross-linking agent. The term “chemical cross-linking agent” refers to an agent which induces chemical cross-linking. The chemical cross-linking agent may be any agent that is capable of inducing a chemical bond between adjacent polymeric chains. For example, the chemical cross-linking agent may be a chemical compound. Examples of chemical compounds that may act as cross-linking agent include, but are not limited to, 1-ethyl-3-[3-dimethylaminopropyl]carbodiimide hydrochloride (EDC), vinylamine, 2-aminoethyl methacrylate, 3-aminopropyl methacrylamide, ethylene diamine, ethylene glycol dimethacrylate, methymethacrylate, N,N′-methylene-bisacrylamide, N,N′-methylene-bis-methacrylamide, diallyltartardiamide, allyl(meth)acrylate, lower alkylene glycol di(meth)acrylate, poly lower alkylene glycol di(meth)acrylate, lower alkylene di(meth)acrylate, divinyl ether, divinyl sulfone, di- or trivinylbenzene, trimethylolpropane tri(meth)acrylate, pentaerythritol tetra(meth)acrylate, bisphenol A di(meth)acrylate, methylenebis(meth)acrylamide, triallyl phthalate, diallyl phthalate, transglutaminase, derivatives thereof or mixtures thereof.

In some embodiments, the hydrogel-forming agents are themselves capable of chemical or physical cross-linking without using a cross-linking agent.

Besides the above-mentioned, the hydrogel-forming agents may be cross-linked using a cross-linking agent in the form of an electromagnetic wave. The cross-linking may be carried out using an electromagnetic wave, such as gamma or ultraviolet radiation, which may cause the polymeric chains to cross-link and form a three-dimensional matrix, thereby entrapping water molecules to form a hydrogel.

Therefore, choice of cross-linking agent is dependent on the type of polymeric chain and functional group present, and a person skilled in the art would be able to choose the appropriate type of cross-linking agent accordingly.

In various embodiments, the hydrogel-forming agent consists essentially of a physically cross-linkable polymer. In some embodiments, the hydrogel-forming agent comprises alginate. Polysaccharides are carbohydrates which may be hydrolyzed to two or more monosaccharide molecules. They may contain a backbone of repeating carbohydrate i.e. sugar unit. In certain aspects the hydrogel comprises polysaccharides. Examples of polysaccharides include, but are not limited to, alginate, agarose, chitosan, dextran, starch, and gellan gum. Glycosaminoglycans are polysaccharides containing amino sugars as a component. Examples of glycosaminoglycans include, but are not limited to, hyaluronic acid, chondroitin sulfate, dermatin sulfate, keratin sulfate, dextran sulfate, heparin sulfate, heparin, glucuronic acid, iduronic acid, galactose, galactosamine, and glucosamine.

Liposome Alginate Microspheres. Alginate is a polysaccharide which forms a hardened gel matrix in the presence of divalent cations such as calcium and barium. Microspheres constructed from alginate have been investigated for the delayed release of therapeutic agents from the alginate matrix. Specifically, low molecular weight molecules (such as doxorubicin) can escape from the spheres and to the target tissue.

Microparticles produced by standard production methods frequently have a wide particle size distribution, lack uniformity, fail to provide adequate release kinetics or other properties, and are difficult and expensive to produce. In addition, the microparticles may be large and tend to form aggregates, requiring a size selection process to remove particles considered to be too large for administration to patients by injection or inhalation. This requires sieving and results in product loss. Certain embodiments described herein use an ultrasonic nozzle or nebulizer to produce liposome-containing microspheres. An ultrasonic nebulizer uses high-frequency electrical energy to create vibrational, mechanical energy, typically employing a piezoelectric transducer. This energy is transmitted to the liquid or formulation to form microspheres either directly or through a coupling fluid, creating an aerosol containing microspheres, which are subsequently cured or cross-linked. Typically, ultrasonic energy disrupts the association of lipids forming a liposome. The liposomes resist the disruptive effects of ultrasound remaining intact during production processes resulting in the formation of smaller liposome-containing alginate microspheres.

In certain aspects, liposome-containing alginate microspheres (LAMs) are produced by spraying a liposome/alginate solution (liquid or feed source) into a curing solution having an alginate cross-linker. Typically, a liquid is supplied by powered pumps to simple or complex orifice nozzles that atomize the liquid stream into spray droplets that are cross-linked when exposed to the curing solution. Nozzles are often selected primarily on the desired range of flow rates needed and secondarily on the range of liquid droplet size. Any spray atomizer that can produce droplets from the liquids described herein can be used. Suitable spray atomizers include two-fluid nozzles, single fluid nozzles, ultrasonic nozzles such as the Sono-Tek™ ultrasonic nozzle, rotary atomizers or vibrating orifice aerosol generators (VOAG), and the like. In certain aspects, the nozzle is an ultrasonic nozzle, a 1 Hz to about 100 kHz nozzle. In one particular aspect the nozzle is a 25 kHz nozzle. In certain aspects, the spray atomizer can have one or more of the following specifications. (a) a 25 kHz to 180 kHz nozzle, in particular a 25 kHz nozzle. (b) a 1 to 10 W generator, in particular a 5.0 W generator. (c) a pump capable of a flow rate of 0.1 to 1.0 ml/min, in particular 0.5 ml/min (microbore may be necessary for a flow rate this low). The curing solution can be positioned to receive the atomized liquid. The distance between the nozzle and the curing solution can be varied between 1 to 10 cm, in particular 4 cm. the system can be activated for the entirety of nozzle usage. The generator can be activated and the pump can form liposome-containing alginate microspheres (LAMs). Microspheres can be incubated at room temp (e.g., 20 to 30° C.) in the curing solution (e.g., CaCl₂ solution) for 1 to 10 minutes, in particular 5 minutes. In certain aspects, the microspheres can be spun down, for example at 1000-1200 rpm. The microsphere solution can be passed through a 100 μm-pore stainless steel mesh for exclusion of any clumping that may have occurred during the cross-linking or centrifugation. These LAMs can be used for post-loading and intraarterial administration. In certain aspects, the microspheres can be visualized under light microscopy, and dosimeter can be used post-loading to measure radioactivity retention in those LAMs loaded with radioactive materials.

Certain embodiments are directed to LAMs having a diameter of 1, 10, 20, 30, 40, 50, 60, 70, 80, 90, 100, 150, 200, 300, 350, 400, 450, to 500 μm, including all values and ranges there between (in certain aspects any of the values or subranges can be specifically excluded). In certain aspects, the LAMs have an average diameter of 20, 30, 40 to 50, 60, 70 80 μm, including all values and ranges there between. In certain aspects the ratio of liposome to alginate (w/w or v/v) is 4:1, 3:1, 2:1, 1:1, 1:2, 1:3, 1:4, including all ratios and ranges there between (in certain aspects any of the values or subranges can be specifically excluded). In certain aspects, the LAM comprises 10 to 80 weight percent liposome/lipid, 10 to 80 weight percent alginate solution, 0.01 to 5 weight percent alginate cross-linker, and 1 to 30 weight percent therapeutic and/or imaging agent.

Chemoembolization or radioembolization are cancer treatments in which particles are delivered to a tumor through the bloodstream. The particles lodge in the tumor and provide a therapeutic, chemotherapy or radiation, that kills cancer cells.

### Liposomes

Liposome loaded Microspheres or Liposome Alginate microspheres (LAMs) provide a more controlled mechanism of sustained release given that eventual rupture of liposomes drives the release of drug rather than current agents which depend on disruption of weak nonspecific bonds. The disruption of a liposome's lipid bilayer can be dependent on a transition temperature. In certain aspects, LAMs employed for radionuclide therapy are loaded with DSPC, having a transition temperature of 55° C. A LAM designed for drug elution could employ a lipid such as DPPC with a transition temperature of 41° C. (closer to physiologic temperature of 37° C.) A sustained elution would most likely be the result of incorporating certain lipids at certain ratios into LAMs.

Selection of the appropriate lipids for liposome composition is governed by the factors of: (1) liposome stability, (2) phase transition temperature, (3) charge, (4) non-toxicity to mammalian systems, (5) encapsulation efficiency, (6) lipid mixture characteristics, and the like. The vesicle-forming lipids preferably have two hydrocarbon chains, typically acyl chains, and a head group, either polar or nonpolar. The hydrocarbon chains may be saturated or have varying degrees of unsaturation. There are a variety of synthetic vesicle-forming lipids and naturally-occurring vesicle-forming lipids, including the sphingolipids, ether lipids, sterols, phospholipids, phosphoglycerides, and glycolipids (e.g., cerebrosides and gangliosides).

Phosphoglycerides include phospholipids such as phosphatidylcholine, phosphatidylethanolamine, phosphatidic acid, phosphatidylinositol, phosphatidylserine phosphatidylglycerol and diphosphatidylglycerol (cardiolipin), where the two hydrocarbon chains are typically between about 14-22 carbon atoms in length, and have varying degrees of unsaturation. As used herein, the abbreviation “PC” stands for phosphatidylcholine, and “PS” stand for phosphatidylserine. Lipids containing either saturated and unsaturated fatty acids are widely available to those of skill in the art. Additionally, the two hydrocarbon chains of the lipid may be symmetrical or asymmetrical. The above-described lipids and phospholipids whose acyl chains have varying lengths and degrees of saturation can be obtained commercially or prepared according to published methods.

Phosphatidylcholines include, but are not limited to dilauroyl phophatidylcholine, dimyristoylphophatidylcholine, dipalmitoylphophatidylcholine, distearoylphophatidyl-choline, diarachidoylphophatidylcholine, dioleoylphophatidylcholine, dilinoleoyl-phophatidylcholine, dierucoylphophatidylcholine, palmitoyl-oleoyl-phophatidylcholine, egg phosphatidylcholine, myristoyl-palmitoylphosphatidylcholine, palmitoyl-myristoyl-phosphatidylcholine, myristoyl-stearoylphosphatidylcholine, palmitoyl-stearoyl-phosphatidylcholine, stearoyl-palmitoylphosphatidylcholine, stearoyl-oleoyl-phosphatidylcholine, stearoyl-linoleoylphosphatidylcholine and palmitoyl-linoleoyl-phosphatidylcholine. Asymmetric phosphatidylcholines are referred to as 1-acyl, 2-acyl-sn-glycero-3-phosphocholines, wherein the acyl groups are different from each other. Symmetric phosphatidylcholines are referred to as 1,2-diacyl-sn-glycero-3-phosphocholines. As used herein, the abbreviation “PC” refers to phosphatidylcholine. The phosphatidylcholine 1,2-dimyristoyl-sn-glycero-3-phosphocholine is abbreviated herein as “DMPC.” The phosphatidylcholine 1,2-dioleoyl-sn-glycero-3-phosphocholine is abbreviated herein as “DOPC.” The phosphatidylcholine 1,2-dipalmitoyl-sn-glycero-3-phosphocholine is abbreviated herein as “DPPC.”

In general, saturated acyl groups found in various lipids include groups having the trivial names propionyl, butanoyl, pentanoyl, caproyl, heptanoyl, capryloyl, nonanoyl, capryl, undecanoyl, lauroyl, tridecanoyl, myristoyl, pentadecanoyl, palmitoyl, phytanoyl, heptadecanoyl, stearoyl, nonadecanoyl, arachidoyl, heneicosanoyl, behenoyl, trucisanoyl and lignoceroyl. The corresponding IUPAC names for saturated acyl groups are trianoic, tetranoic, pentanoic, hexanoic, heptanoic, octanoic, nonanoic, decanoic, undecanoic, dodecanoic, tridecanoic, tetradecanoic, pentadecanoic, hexadecanoic, 3,7,11,15-tetramethylhexadecanoic, heptadecanoic, octadecanoic, nonadecanoic, eicosanoic, heneicosanoic, docosanoic, trocosanoic and tetracosanoic. Unsaturated acyl groups found in both symmetric and asymmetric phosphatidylcholines include myristoleoyl, palmitoleyl, oleoyl, elaidoyl, linoleoyl, linolenoyl, eicosenoyl and arachidonoyl. The corresponding IUPAC names for unsaturated acyl groups are 9-cis-tetradecanoic, 9-cis-hexadecanoic, 9-cis-octadecanoic, 9-trans-octadecanoic, 9-cis-12-cis-octadecadienoic, 9-cis-12-cis-15-cis-octadecatrienoic, 11-cis-eicosenoic and 5-cis-8-cis-11-cis-14-cis-eicosatetraenoic.

Phosphatidylethanolamines include, but are not limited to dimyristoyl-phosphatidylethanolamine, dipalmitoyl-phosphatidylethanolamine, di stearoyl-phosphatidylethanolamine, dioleoyl-phosphatidylethanolamine and egg phosphatidylethanolamine. Phosphatidylethanolamines may also be referred to under IUPAC naming systems as 1,2-diacyl-sn-glycero-3-phosphoethanolamines or 1-acyl-2-acyl-sn-glycero-3-phosphoethanolamine, depending on whether they are symmetric or assymetric lipids.

Phosphatidic acids include, but are not limited to dimyristoyl phosphatidic acid, dipalmitoyl phosphatidic acid and dioleoyl phosphatidic acid. Phosphatidic acids may also be referred to under IUPAC naming systems as 1,2-diacyl-sn-glycero-3-phosphate or 1-acyl-2-acyl-sn-glycero-3-phosphate, depending on whether they are symmetric or assymetric lipids.

Phosphatidylserines include, but are not limited to dimyristoyl phosphatidylserine, dipalmitoyl phosphatidylserine, dioleoylphosphatidylserine, distearoyl phosphatidylserine, palmitoyl-oleylphosphatidylserine and brain phosphatidylserine. Phosphatidylserines may also be referred to under IUPAC naming systems as 1,2-diacyl-sn-glycero-3-[phospho-L-serine] or 1-acyl-2-acyl-sn-glycero-3-[phospho-L-serine], depending on whether they are symmetric or assymetric lipids. As used herein, the abbreviation “PS” refers to phosphatidylserine.

Phosphatidylglycerols include, but are not limited to dilauryloylphosphatidylglycerol, dipalmitoylphosphatidylglycerol, distearoylphosphatidylglycerol, dioleoyl-phosphatidylglycerol, dimyristoylphosphatidylglycerol, palmitoyl-oleoyl-phosphatidylglycerol and egg phosphatidylglycerol. Phosphatidylglycerols may also be referred to under IUPAC naming systems as 1,2-diacyl-sn-glycero-3-[phospho-rac-(1-glycerol)] or 1-acyl-2-acyl-sn-glycero-3-[phospho-rac-(1-glycerol)], depending on whether they are symmetric or assymetric lipids. The phosphatidylglycerol 1,2-dimyristoyl-sn-glycero-3-[phospho-rac-(1-glycerol)] is abbreviated herein as “DMPG”. The phosphatidylglycerol 1,2-dipalmitoyl-sn-glycero-3-(phospho-rac-1-glycerol) (sodium salt) is abbreviated herein as “DPPG”.

Suitable sphingomyelins include, but are not limited to brain sphingomyelin, egg sphingomyelin, dipalmitoyl sphingomyelin, and distearoyl sphingomyelin.

Other suitable lipids include glycolipids, sphingolipids, ether lipids, glycolipids such as the cerebrosides and gangliosides, and sterols, such as cholesterol or ergosterol. As used herein, the term cholesterol is sometimes abbreviated as “Chol.” Additional lipids suitable for use in liposomes are known to persons of skill in the art.

In certain aspects the overall surface charge of the liposome can be varied. In certain embodiments anionic phospholipids such as phosphatidylserine, phosphatidylinositol, phosphatidic acid, and cardiolipin are used. Neutral lipids such as dioleoylphosphatidyl ethanolamine (DOPE) may be used. Cationic lipids may be used for alteration of liposomal charge, as a minor component of the lipid composition or as a major or sole component. Suitable cationic lipids typically have a lipophilic moiety, such as a sterol, an acyl or diacyl chain, and where the lipid has an overall net positive charge. Preferably, the head group of the lipid carries the positive charge.

One of skill in the art will select vesicle-forming lipids that achieve a specified degree of fluidity or rigidity. The fluidity or rigidity of the liposome can be used to control factors such as the stability of the liposome or the rate of release of an entrapped agent. Liposomes having a more rigid lipid bilayer, or a liquid crystalline bilayer, are achieved by incorporation of a relatively rigid lipid. The rigidity of the lipid bilayer correlates with the phase transition temperature of the lipids present in the bilayer. Phase transition temperature is the temperature at which the lipid changes physical state and shifts from an ordered gel phase to a disordered liquid crystalline phase. Several factors affect the phase transition temperature of a lipid including hydrocarbon chain length and degree of unsaturation, charge and headgroup species of the lipid. Lipid having a relatively high phase transition temperature will produce a more rigid bilayer. Other lipid components, such as cholesterol, are also known to contribute to membrane rigidity in lipid bilayer structures. Cholesterol is widely used by those of skill in the art to manipulate the fluidity, elasticity and permeability of the lipid bilayer. It is thought to function by filling in gaps in the lipid bilayer. In contrast, lipid fluidity is achieved by incorporation of a relatively fluid lipid, typically one having a lower phase transition temperature. Phase transition temperatures of many lipids are tabulated in a variety of sources.

In certain aspects, liposomes are made from endogenous phospholipids such as dimyristoyl phosphatidylcholine (DMPC) and dimyristoyl phosphatidylglycerol (DMPG), phosphatidyl serine, phosphatidyl choline, dioleoyphosphatidyl choline [DOPC], cholesterol (CHOL) and cardiolipin.

Liposome Loading Efficiency. The loading efficiency of loading methods for liposomes can be measured by use of conventional methods in the art including ion-exchange chromatography, radio thin layer chromatography (radio-TLC), dialysis, or size exclusion chromatography (SEC) which can separate free radioactive metal ions or free radiolabeled complexes from liposome encapsulated radionuclides. When using SEC, the amount of radioactivity retained in liposomes compared to the amount of free radioactive metal ions or free radiolabeled complexes can be determined by monitoring the elution profile during SEC and measuring the radioactivity with a radioactivity detector, or measuring the concentration of the metal entity using inductively coupled plasma mass spectrometry (ICP-MS), inductively coupled plasma atomic emission spectroscopy (ICP-AES) or inductively coupled plasma optical emission spectrometry (ICP-OES). The radioactivity measured in the eluted fractions containing liposomes compared to eluted fractions not containing liposomes can be used to determine the loading efficiency by calculating the percentage of radioactivity retained in liposomes. Likewise, the amount of radioactivity bound in liposomes can be compared to the amount of radioactivity not entrapped in liposomes to obtain a measure of the loading efficiency when using other conventional methods known in the art.

The methods of the present invention ensure that a high amount of the radionuclides used in preparation will be entrapped within the liposomes present in the microsphere. The encapsulation or loading efficiency, defined as encapsulated (internal) amount of the agent or complex being loaded in the liposome(s) divided by the initial amount external liposome multiplied by 100. In one embodiment of the present method the efficiency of loading can higher than 10%, such as in the range of 10%-100%, such as higher than 15%, such as higher than 20%, such as higher than 25%, such as higher than 30%, such as higher than 35%, for example higher than 40%, such as higher than 50%, for example higher than 60%, such as higher than 65%, for example higher than 70%, such as higher than 75%, for example higher than 80%, such as higher than 85%, for example higher than 90%, such as higher than 95%, or such as higher than 96%, or such as higher than 97%, or such as higher than 98%, or such as higher than 99% or such as higher than 99.5% or such as higher than 99.9%. In another embodiment of the present invention the efficiency of loading when using the methods of the present invention is higher than 30% when assayed using size exclusion chromatography (SEC, described in examples), ion-exchange chromatography or dialysis, such as 30% to 100%, including 55% to 100% loading efficiency, 80% to 100% loading efficiency, and 95% to 100% loading efficiency.

Preferably, the efficiency of loading of the methods according to the present invention is in the range of 55% to 100% such as in the range of 80% to 100%, more preferably in the range of 95% to 100%, such as between 95% to 97%, or such as between 97% to 99.9% loading efficiency.

Agent-Entrapping of Loading Component. The agent-entrapping component of the present invention or the method of the present invention may be a chelating agent that forms a chelating complex with the transition metal or the radiolabeled agent, such as the radionuclide.

When a chelator (such as for example DOTA) is present in the aqueous phase of the liposome interior, the equilibrium between the exterior and the interior of the liposome is shifted since metal ions that pass the membrane barrier are effectively removed from the inner membrane leaflet due to tight binding to the chelator. The very effective complex formation of the metal ion with the chelator renders the free metal ion concentration in the liposome interior negligible and loading proceeds until all metal ions have been loaded into the liposome or equilibrium has been reached. If excess of chelator is used, the metal ion concentration in the liposomes will be low at all stages during loading and the trans-membrane gradient will be defined by the free metal ion concentration on the exterior of the liposomes.

According to the present invention, chelators may be selected from the group comprising 1,4,7,10-tetraazacyclododecane-1,4,7,10-tetraacetic acid (DOTA) and derivatives thereof; 1,4,8,11-tetraazacyclotetradecane (cyclam) and derivatives thereof; 1,4,7,10-tetraazacyclododecane (cycl en) and derivatives thereof; 1,4-ethano-1,4,8,11-tetraazacyclotetradecane (et-cyclam) and derivatives thereof; 1,4,7,11-tetraazacyclotetradecane (isocyclam) and derivatives thereof; 1,4,7,10-tetraazacyclotridecane ([13]aneN4) and derivatives thereof; 1,4,7,10-tetraazacyclododecane-1,7-diacetic acid (DO2A) and derivatives thereof; 1,4,7,10-tetraazacyclododecane-1,4,7-triacetic acid (DO3A) and derivatives thereof; 1,4,7,10-tetraazacyclododecane-1,7-di(methanephosphonic acid) (DO2P) and derivatives thereof; 1,4,7,10-tetraazacyclododecane-1,4,7-tri(methanephosphonic acid) (DO3P) and derivatives thereof; 1,4,7,10-tetraazacyclododecane-1,4,7,10-tetra(methanephosphonic acid) (DOTP) and derivatives thereof; ethylenediaminetetraacetic acid (EDTA) and derivatives thereof; diethylenetriaminepentaacetic acid (DTPA) and derivatives thereof; 1,4,8,11-tetraazacyclotetradecane-1,4,8,11-tetraacetic acid (TETA) and derivatives thereof, or other adamanzanes and derivates thereof.

In another embodiment, the agent-entrapping component according to the present invention may be a substance that has the ability to reduce other substances, thus referred to as a reducing agent. Examples of reducing agents comprise ascorbic acid, glucose, fructose, glyceraldehyde, lactose, arabinose, maltose and acetol.

In a further embodiment, an agent-entrapping component within the scope of the present invention or the method of present invention may be a substance with which the radionuclide or metal entity forms a low solubility salt.

In one embodiment of the present invention or the method of the present invention the agent-entrapping component is a chelator selected from the group consisting of macrocyclic compounds comprising adamanzanes; 1,4,7,10-tetraazacyclododecane ([12]aneN4) or a derivative thereof; 1,4,7,10-tetraazacyclotridecane ([13]aneN4) or a derivative thereof; 1,4,8,11-tetraazacyclotetradecane ([14]aneN4) or a derivative thereof; 1,4,8,12-tetraazacyclopentadecane ([15]aneN4) or a derivative thereof; 1,5,9,13-tetraazacyclohexadecane ([16]aneN4) or a derivative thereof and other chelators capable of binding metal ions such as ethylene-diamine-tetraacetic-acid (EDTA) or a derivative thereof, diethylene-triamine-penta-acetic acid (DTPA) or a derivative thereof.

In one embodiment of the present invention or the method of the present invention the agent-entrapping component is a chelator selected from the group consisting of 1,4-ethano-1,4,8,11-tetraazacyclotetradecane (et-cyclam) or a derivative thereof; 1,4,7,11-tetraazacyclotetradecane (iso-cyclam) or a derivatives thereof; 1,4,7,10-tetraazacyclododecane-1,4,7,10-tetraacetic acid (DOTA) or a derivative thereof 2-(1,4,7,10-tetraazacyclododecan-1-yl)acetate (DO1A) or a derivative thereof 2,2′-(1,4,7,10-tetraazacyclododecane-1,7-diyl) diacetic acid (DO2A) or a derivative thereof 2,2′,2″-(1,4,7,10-tetraazacyclododecane-1,4,7-triyl)triacetic acid (DO3A) or a derivative thereof; 1,4,7,10-tetraazacyclododecane-1,4,7,10-tetra(methanephosphonic acid) (DOTP) or a derivative thereof; 1,4,7,10-tetraazacyclododecane-1,7-di(methanephosphonic acid) (DO2P) or a derivative thereof; 1,4,7,10-tetraazacyclododecane-1,4,7-tri(methanephosphonic acid) (DO3P) or a derivative thereof; 1,4,8,11-15 tetraazacyclotetradecane-1,4,8,11-tetraacetic acid (TETA) or a derivative thereof; 2-(1,4,8,11-tetraazacyclotetradecane-1-yl)acetic acid (TE1A) or a derivative thereof; 2,2′-(1,4,8,11-tetraazacyclotetradecane-1,8-diyl)diacetic acid (TE2A) or a derivative thereof and other adamanzanes or derivates thereof.

In one embodiment of the present invention or the method of the present invention the agent-entrapping component is selected from the group consisting of 1,4,7,10-tetraazacyclododecane-1,4,7,10-tetraacetic acid (DOTA) or a derivative thereof, 1,4,8,11-15 tetraazacyclotetradecane-1,4,8,11-tetraacetic acid (TETA) or a derivative thereof, 1,4,7,10-tetraazacyclododecane-1,4,7,10-tetra(methanephosphonic acid) (DOTP), cyclam and cyclen.

In a particularly important embodiment of the present invention or method of the present invention, the agent-entrapping component is 1,4,7,10-tetraazacyclododecane-1,4,7,10-tetraacetic acid (DOTA).

Ionophores can be characterized as ion-transporters, lipophilic chelators, channel formers, lipophilic complexes etc. In general an ionophore can be defined as a lipid-soluble molecule that transports ions across the lipid bilayer of cell membranes or liposomes. Ionophores are used to increase permeability of lipid membranes to ions and facilitate transfer of molecules through, into and out of the membrane. There are general two broad classifications of ionophores, where one is; chemical compounds, mobile carriers or lipophilic chelators that bind or chelate to a particular ion or molecule, shielding its charge from the surrounding environment, and thus facilitating its crossing of the hydrophobic interior of the lipid membrane. The second classification is; channel formers that introduce a hydrophilic pore into the membrane, allowing molecules or metal ions to pass through while avoiding contact with the hydrophobic interior of the membrane.

In conventional methods using ionophores, or other components capable of transporting ions or loading of nanoparticles, the resulting nanoparticles comprise small amounts of the ion-transporter or ionophore used in the loading procedure. The nanoparticles provided by the present invention are prepared without the use of an ion-transporter such as an ionophore. Thus, the present invention relates to nanoparticle compositions, which do not comprise ion-transporters or ionophores.

In another embodiment of the present invention, the nanoparticle compositions as defined herein do not comprise any added ionophores.

Ion-transporters or ionophoric compounds which are not comprised in the nanoparticles of the present invention may be selected from the group of 8-hydroxyquinoline (oxine); 8-hydroxyquinoline β-D-galactopyranoside; 8-hydroxyquinoline β-D-glucopyranoside; 8-hydroxyquinoline glucuronide; 8-hydroxyquinoline-5-sulfonic acid; 8-hydroxyquinoline-β-D-glucuronide sodium salt; 8-quinolinol hemisulfate salt; 8-quinolinol N-oxide; 2-amino-8-quinolinol; 5,7-dibromo-8-hydroxyquinoline; 5,7-dichloro-8-hydroxyquinoline; 5,7-diiodo-8-hydroxyquinoline; 5,7-dimethyl-8-quinolinol; 5-amino-8-hydroxyquinoline dihydrochloride; 5-chloro-8-quinolinol; 5-nitro-8-hydroxyquinoline; 7-bromo-5-chloro-8-quinolinol; N-butyl-2,2′-imino-di(8-quinolinol); 8-hydroxyquinoline benzoate; 2-benzyl-8-hydroxyquinoline; 5-chloro-8-hydroxyquinoline hydrochloride; 2-methyl-8-quinolinol; 5-chloro-7-iodo-8-quinolinol; 8-hydroxy-5-nitroquinoline; 8-hydroxy-7-iodo-5-quinolinesulfonic acid; 5,7-dichloro-8-hydroxy-2-methyl quinoline, and other quinolines (1-azanaphthalene, 1-benzazine) consisting chemical compounds and derivatives thereof. In one embodiment the ionophoric compound is selected from the group consisting of: 8-hydroxyquinoline (oxine); 8-hydroxyquinoline β-D-galactopyranoside; 8-hydroxyquinoline 3-D-glucopyranoside; 8-hydroxyquinoline glucuronide; 8-hydroxyquinoline-5-sulfonic acid; 8-hydroxyquinoline-β-D-glucuronide sodium salt; 8-quinolinol hemisulfate salt; 8-quinolinol N-oxide; 2-amino-8-quinolinol; 5,7-dibromo-8-hydroxyquinoline; 5,7-dichloro-8-hydroxyquinoline; 5,7-diiodo-8-hydroxyquinoline; 5,7-dimethyl-8-quinolinol; 5-amino-8-hydroxyquinoline dihydrochloride; 5-chloro-8-quinolinol; 5-nitro-8-hydroxyquinoline; 7-bromo-5-chloro-8-quinolinol; N-butyl-2,2′-imino-di(8-quinolinol); 8-hydroxyquinoline benzoate; 2-benzyl-8-hydroxyquinoline; 5-chloro-8-hydroxyquinoline hydrochloride; 2-methyl-8-quinolinol; 5-chloro-7-iodo-8-quinolinol; 8-hydroxy-5-nitroquinoline; 8-hydroxy-7-iodo-5-quinolinesulfonic acid; 5,7-dichloro-8-hydroxy-2-methylquinoline, and other quinolines (1-azanaphthalene, 1-benzazine) consisting chemical compounds and derivatives thereof.

Ion-transporters or ionophoric compounds which are not comprised in the nanoparticles or used in the methods of the present invention may additionally be selected from the group consisting of 2-hydroxyquinoline-4-carboxylic acid; 6-chloro-2-hydroxyquinoline; 8-chloro-2-hydroxyquinoline; carbostyril 124; carbostyril 165; 4,6-dimethyl-2-hydroxyquinoline; 4,8-dimethyl-2-hydroxyquinoline; or other 2-quinolinol compounds 8-hydroxyquinoline (oxine); 8-hydroxyquinoline β-D-galactopyranoside; 8-hydroxyquinoline 3-D-glucopyranoside; 8-hydroxyquinoline glucuronide; 8-hydroxyquinoline-5-sulfonic acid; 8-hydroxyquinoline-β-D-glucuronide sodium salt; 8-quinolinol hemisulfate salt; 8-quinolinol N-oxide; 2-amino-8-quinolinol; 5,7-dibromo-8-hydroxyquinoline; 5,7-dichloro-8-hydroxyquinoline; 5,7-diiodo-8-hydroxyquinoline; 5,7-dimethyl-8-quinolinol; 5-amino-8-hydroxyquinoline dihydrochloride; 5-chloro-8-quinolinol; 5-nitro-8-hydroxyquinoline; 7-bromo-5-chloro-8-quinolinol; N-butyl-2,2′-imino-di(8-quinolinol); 8-hydroxyquinoline benzoate; 2-benzyl-8-hydroxyquinoline; 5-chloro-8-hydroxyquinoline hydrochloride; 2-methyl-8-quinolinol; 5-chloro-7-iodo-8-quinolinol; 8-hydroxy-5-nitroquinoline; 8-hydroxy-7-iodo-5-quinolinesulfonic acid; 5,7-dichloro-8-hydroxy-2-methylquinoline, and other quinolines (1-azanaphthalene, 1-benzazine) consisting chemical compounds and derivatives thereof, [6S-[6α(2S*,3S*), 8β(R*),9β, 11.alpha]]-5-(methylamino)-2-[[3,9,11-trimethyl-8-[1-methyl-2-oxo-2-(1H-pyrrol2-yl)ethyl]-1,7-dioxaspiro[5.5]undec-2-yl]methyl]-4-benzoxazolecarboxylic acid (also called A23187), HMPAO (hexamethyl propylene amine oxime, HYNIC (6-Hydrazinopyridine-3-carboxylic acid), BMEDA (N—N-bis(2-mercaptoethyl)-N′,N′-diethylethylenediamine), DISIDA (diisopropyl iminodiacetic acid, phthaldialdehyde and derivatives thereof, 2,4-dinitrophenol and derivatives thereof, di-benzo-18-crown-6 and derivatives thereof, o-xylylenebis(N,N-diisobutyldithiocarbamate) and derivatives thereof, N,N,N′,N′-Tetracyclohexyl-2,2′-thiodiacetamide and derivates thereof, 2-(1,4,8,11-Tetrathiacyclotetradec-6-yloxy)hexanoic acid, 2-(3,6,10,13-Tetrathiacyclotetradec-1-oxy)hexanoic acid and derivates thereof, N,N-bis(2-mercaptoethyl)-N′,N′-diethylethylenediamine and derivates thereof, beauvericin, enniatin, gramicidin, ionomycin, lasalocid, monesin, nigericin, nonactin, nystatin, salinomycin, valinomycin, pyridoxal isonicotinoyl hydrazone (PIH), salicylaldehyde isonicotinoyl hydrazone (SIH), 1,4,7-trismercaptoethyl-1,4,7-triazacyclononane, N,N′,N″-tris(2-mercaptoethyl)-1,4,7-triazacyclononane, monensis, DP-b99, DP-109, BAPTA, pyridoxal isonicotinoyl hydrazone (PIH), alamethicin, di-2-pyridylketone thiosemicarbazone (HDpT), carbonyl cyanide m-chlorophenyl hydrazone (CCCP), lasalocid A (X-537A), 5-bromo derivative of lasalocid; cyclic depsipeptides; cyclic peptides: DECYL-2; N,N,N′,N′-tetrabutyl-3,6-dioxaoctanedi[thioamide]); N,N,N′,N′-tetracyclohexyl-3-oxa-pentanediamide; N,N-dicyclohexyl-N′,N′-dioctadecyl-diglycolic-diamide; N,N′-diheptyl-N,N′-dimethyl-1,-butanediamide; N,N″-octamethylene-bis[N′-heptyl-N′-methyl-malonamide; N,N-dioctadecyl-N′,N′-dipropyl-3,6-dioxaoctanediamide; N-[2-(1H-pyrrolyl-methyl)]-N′-(4-penten-3-on-2)-ethane-1,2-diamine (MRP20); and antifungal toxins; avenaciolide or derivatives of the above mentioned ionophores, as well as the ionophores described in WO2011/006510 and other ionophores described in the art.

pH gradient loadable agents are agents with one or more ionisable moieties such that the neutral form of the ionisable moiety allows the metal entities to cross the liposome membrane and conversion of the moiety to a charged form causes the metal entity to remain encapsulated within the liposome are also regarded as ionophores according to the present invention. Ionisable moieties may comprise, but are not limited to comprising, amine, carboxylic acid and hydroxyl groups. pH gradient loadable agents that load in response to an acidic interior may comprise ionisable moieties that are charged in response to an acidic environment whereas drugs that load in response to a basic interior comprise moieties that are charged in response to a basic environment. In the case of a basic interior, ionisable moieties including but not limited to carboxylic acid or hydroxyl groups may be utilized.

The interior pH of the nanoparticles according to the present invention can be controlled to lie in a specific range wherein the features of the nanoparticle are optimized.

In one embodiment of the present invention or the method of the present invention, the interior pH of the liposome composition is controlled, thus achieving a desired protonation state of the agent-entrapping component and/or the ionophore, thereby securing efficient loading and entrapment of the radionuclide.

In a preferred embodiment of the present invention or the method of the present invention, the interior pH of the liposome composition is controlled, thus achieving a desired protonation state of the agent-entrapping component, thereby securing efficient loading and entrapment of the radionuclide.

In another embodiment of the disclosed method for producing a nanoparticle composition loaded with a copper isotope, the interior pH is controlled during synthesis of the nanoparticles in such a way that the interior pH of the nanoparticles is within the range of 1 to 10, such as 1-2, for example 2-3, such as 3-4, for example 4-5, such as 5-6, for example 6-7, such as 7-8, for example 8-9, such as 9-10.

In a preferred embodiment of the present invention, the interior pH of the nanoparticles (liposomes) is in the range of 4 to 8.5, such as 4.0 to 4.5, for example 4.5 to 5.0, such as 5.0 to 5.5 for example 5.5 to 6.0, such as 6.0 to 6.5, for example 6.5 to 7.0, such as 7.0 to 7.5, for example 7.5 to 8.0, such as 8.0 to 8.5.

In another embodiment of the present invention, the interior pH of the nanoparticles according to the present invention is optimized in order to prolong the stability of the nanoparticles. Such improved stability can for example lead to a longer shelf-life or a wider range of possible storage temperatures and thereby facilitate the use of the nanoparticles. The improved stability can be obtained, for example because the interior pH leads to an increased stability of the vesicle forming components forming a vesicle, due to increased stability of the agent-entrapping component with or without the entrapped radionuclides or due to improved stability of other features of the nanoparticles. An interior pH which is optimized for improved stability may be within the range of 1 to 10, such as 1-2, for example 2-3, such as 3-4, for example 4-5, such as 5-6, for example 6-7, such as 7-8, for example 8-9, such as 9-10.

In a preferred embodiment of the present invention, the interior pH which leads to an improved stability of the nanoparticles is in the range of 4 to 8.5, such as 4.0 to 4.5, for example 4.5 to 5.0, such as 5.0 to 5.5 for example 5.5 to 6.0, such as 6.0 to 6.5, for example 6.5 to 7.0, such as 7.0 to 7.5, for example 7.5 to 8.0, such as 8.0 to 8.5.

### Methods of Administration and Treatment

Currently, Transarterial chemoembolization (TACE) is a similar practice to TARE in which drug eluting beads loaded with chemotherapeutic agents (most notably doxorubicin) are delivered to hepatic tumors. Microspheres formed from polyvinyl alcohol are modified to carry nonspecific binding groups which allow for drug eluting properties to these microspheres; however, the drug loading capacity and diffuse rate are suboptimal given the nonspecific binding mechanism. A mechanism for a more sustained release for TACE would be highly favored.

LAMs describe herein are candidates for TACE in addition to TARE. Theoretically, given that BMEDA and Doxorubicin are amphipathic weak bases, they may both undergo the same mechanism of diffusion into microencapsulated pH gradient liposomes.

Embolism Therapy. Methods of tumor arterial embolism include the injection of an embolus into micro-arteries, causing mechanical blocking and inhibiting tumor growth. In certain aspects, the embolus is a liposome alginate microsphere (LAM) as described herein. In certain aspects, the tumors treated are malignant tumors unsuitable for surgical operations. The tumors can be hepatocellularcarcinoma (HCC), renal cancer, tumors in pelvis and head and neck cancer.

Effectiveness of a microsphere for embolism purposes depends on one or more of microsphere diameter, microsphere degradation rate, and therapeutic agent release rate. The microsphere preparations can block micro-vessels that are supporting the cancer or tumor. The embolism can supply a therapeutic agent that is targeted to the tumor, allowing the therapeutic agent to be targetable and controllable. This kind of drug administration is able to improve drug distribution in vivo and enhance pharmacokinetic features, increase bioavailability of drugs, improving treatment effect, and alleviate toxic or side effects.

In certain aspects the radioembolic therapy can be used in combination with a radiation sensitizer. In the present invention, the term “radiation sensitizer” or “radiosensitizer” means a compound which enhances the effect of radiation. Examples of radiation sensitizers include, but are not limited to, nitroimidazoles such as misonidazole, etanidazole, metronidazole, and nimorazole; docetaxel, paclitaxel, idoxuridine, fludarabine, gemcitabine, and taxanes.

### Kit Comprising Post Loaded Liposome-Containing Microspheres

The present invention provides kit of parts for preparation of the Microsphere composition post manufacture, i.e., for post-loading. Such a kit may comprise: a microsphere or LAM composition comprising a liposome loaded microsphere and an agent-entrapping or loading component. In one embodiment, the kit can include an agent to encapsulate or a metal entity such as a radionuclide. In certain aspects the agent to encapsulate is provided separately.

The metal entity or radionuclide is either in storage or delivered from the manufacturer depending on the characteristics of the particular radionuclide. The radionuclide may be delivered in the form of a (lyophilized) salt or an aqueous solution or may be synthesized on the premises using existing production facilities and starting materials. Before administration of the radionuclide-containing nanoparticles, the components of the kit are used in a post-loading procedure described herein.

### EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

A Method for Loading Tc-99M and R-186 into Alginate Microspheres for Radioembolization

Beta-emitting yttrium-90 spheres serve as a staple agent for radioembolization; however, limitations include costly production, shunting from hepatic to pulmonary circulation, and limited post-procedural visualization. The production of Liposomes in Alginate Microsphere (LAMs) which may be loaded with either Tc-99m or Re-186 have been previously described. These microspheres have great implications toward radioembolization applications; however, the inventors propose an improved modality for their production in which pH gradient liposomes are encapsulated in alginate microspheres and subsequentially radiolabeled after production.

Materials and Methods. In brief, pH gradient liposomes were manufactured and microencapsulated in alginate microspheres via ultrasonication atomization. Microsphere diameter was measured via light microscopy. Microspheres were subsequentially incubated with Re-186/Tc-99m-BMEDA complex and then washed to remove unencapsulated radionuclide. Re-186/Tc-99m-BMEDA complex was incubated with alginate microspheres (minus any liposomes) for direct comparison to LAMs using gamma imaging. Tc-LAMs were intra-arterially delivered to an ex vivo bovine kidney perfusion model to assess embolization. Blood pressure and flow rate of the kidney were recorded. Venous return was collected during microsphere delivery. Five minute planar gamma image and SPECT was obtained of the embolized kidney and venous return.

Results. LAMs were constructed with a mean diameter of 49.5 μm (STDV=10.4 μm). Re-LAMs demonstrated a radiolabeling efficiency of 51% whereas alginate sphere with no liposomes retained 15% of dose. 2 ml of 2.98 mCi Tc-LAMs were subsequentially constructed for delivery to the ex vivo kidney. BP was approximately 110/50 with a flow rate of approximately 300 ml/min upon perfusion. The full dose of spheres was nonselectively delivered to the kidney via 3Fr microcatheter. Gamma imaging of venous return demonstrated venous shunting of 3.7% of radioactivity. SPECT demonstrated high activity in the renal cortex with trace dose appreciated along the venous outflow tract.

Conclusion. The method for radiolabeling LAMs after production demonstrated success regarding radioactivity retention and embolization capabilities. The proposed method facilitates the manufacture of the LAMs by radiopharmacies, without sacrificing the stability and radioactive retention of the microspheres.

1. A method for post-manufacture loading of a liposome-containing hydrogel microsphere comprising contacting a microsphere containing a plurality of pH gradient liposomes with a loading complex comprising a therapeutic agent complexed with a loading agent or a diagnostic agent complexed with a loading agent, or any combination thereof, wherein the loading agent is retained in liposome.

2. The method of claim 1, wherein the hydrogel microsphere is a polysaccharide microsphere.

3. The method of claim 2, wherein the polysaccharide microsphere is an alginate microsphere.

4. The method of claim 1, wherein the imaging agent is ^{99m}Tc.

5. The method of claim 1, wherein the therapeutic agent is a chemotherapeutic agent or a radiotherapeutic agent.

6. The method of claim 5, wherein the chemotherapeutic agent is a taxane, epothilones, anthracycline, or vinca alkaloid.

7. The method of claim 5, wherein the radiotherapeutic agent is ¹³¹I, ⁹⁰Y, ¹⁷⁷Lu, ¹⁸⁶Re, ¹⁸⁸Re, ¹²⁵I, or ¹²³I, or any combination thereof.

8. The method of claim 1, wherein the loading agent is BMEDA.

9. A kit for post-loading a liposome-containing hydrogel microsphere comprising (i) a container of hydrogel microspheres and (ii) a loading agent.

10. A liposome-containing microsphere comprising a microsphere encapsulating a plurality of pH gradient liposomes encapsulating a therapeutic agent complexed with a loading agent, diagnostic agent complexed with a loading agent, or any combination thereof, wherein the loading efficiency of a therapeutic agent is 40 to 100%.

11. The liposome-containing microsphere of claim 10, wherein the hydrogel microsphere is a polysaccharide microsphere.

12. The liposome-containing microsphere of claim 11, wherein the polysaccharide microsphere is an alginate microsphere.

13. The liposome-containing microsphere of any one of claims 10 to 12, wherein the liposome is sphingolipids, ether lipids, sterols, phospholipids, phosphoglycerides, or glycolipids

14. The liposome-containing microsphere of claim 10, wherein the imaging agent is ^{99m}Tc.

15. The liposome-containing microsphere of any one of claims 10 to 14, wherein the therapeutic agent is a chemotherapeutic agent or a radiotherapeutic agent.

16. The liposome-containing microsphere of claim 15, wherein the chemotherapeutic agent is a taxane, epothilones, or vinca alkaloid.

17. The liposome-containing microsphere of claim 15, wherein the radiotherapeutic agent is ¹³¹I, ⁹⁰Y, ¹⁷⁷Lu, ¹⁸⁶Re, ¹⁸⁸Re, ¹²⁵I, or ¹²³I, or any combination thereof.

18. The liposome-containing microsphere of any one of claims 10 to 17, wherein the loading agent is BMEDA.

19. A method for performing embolization therapy on a subject having a tumor comprising injecting the liposome-containing microsphere of any one of claims 11 to 18 into the tumor vasculature.

20. A liposome-containing microsphere composition for use in treating or diagnosing a condition in a subject, the liposome-containing microsphere comprising a microsphere encapsulating a plurality of pH gradient liposomes encapsulating a therapeutic agent complexed with a loading agent, diagnostic agent complexed with a loading agent, or a combination thereof, wherein the loading efficiency of a therapeutic agent is 10 to 100%.

21. The liposome-containing microsphere composition of claim 20, wherein the therapeutic agent or diagnostic agent is one or more of ¹³¹I, ⁹⁰Y, ^{99m}Tc, ¹⁷⁷Lu, ¹⁸⁶Re, ¹⁸⁸Re, ¹²¹I, or ¹²³I.

22. A liposome-containing microsphere produced by the method of any one of claim 1 to claim 8.
